(19) United States
(12) Patent Application Publication
(10) Pub. No.: US 2006/0041230 A1
(43) Pub. Date: Feb. 23, 2006

(54) Over-the needle peel-away sheath catheter introducer

(76) Inventor: Jeremy M. Davis, San Antonio, TX (US)

Correspondence Address: Amster, Rothstein & Ebenstein LLP;Attn: Kenneth P. George, Esq., 90 Park Avenue, New York, NY, 10016, US

(21) Appl. No.: 10/920,064

(22) Filed: Aug. 17, 2004

(51) Int. Cl. A61M 5/178

(52) U.S. Cl. 604/160

(57) ABSTRACT

An over-the-needle peel-away sheath catheter introducer for receiving the distal end of a catheter is formed of a hub having left and right halves with a wing extending transversely from each half. The hub defines between these halves a passageway for a catheter, where the passageway has sides defined by opposing wall surfaces of said halves, and top and bottom walls extending between these halves. A funnel is situated at the proximal end of the hub and a sheath is situated at the distal end of the hub. The funnel, hub and sheath have respective lumens which are aligned forming a continuous passageway. The funnel, hub and sheath furthermore have tear lines along their top and bottom surfaces adapting this catheter introducer to be split apart into two halves and discarded after introduction of a catheter therethrough.

### BACKGROUND OF THE INVENTION

1. Field of the Invention

This invention is in the field of peel-away catheter introducers and more particularly in the field of peel-away catheter introducers for neonatal and premature infants whose limbs and veins are extremely small.

2. Prior Art

Catheter introducers are typically used in conjunction with peripherally inserted central catheters (PICC), guide wires or other relatively long, thin and flexible medical devices, to facilitate insertion and placement of catheters or other medical devices into the patients' vasculature. A conventional peel-away catheter introducer, also called a peelable or splittable catheter introducer, comprises a hub with a pair of wings extending transversely of the proximal end of the hub and a sheath extending distally of the distal end of the hub. With such a catheter introducer an introducer needle is positionable through the introducer hub with the sharp distal tip of the needle extending distally of the distal end of the sheath. As used herein, the term proximal refers to a location on the device closest to the clinician using the device. Conversely, the term distal refers to a location on the device farthest from the clinician, such as the distal tip of the device to be inserted into a vein. Furthermore, the hub and sheath components each have a proximal end and a distal end. Some prior art introducers also include a funnel-like element at the proximal end of the hub to facilitate introduction of a needle for venipuncture or introduction of a catheter or guide wire thereafter.

When using a conventional peel-away catheter introducer the clinician grasps the needle hub along with the introducer hub and proceeds to insert the distal portions of the needle and introducer sheath at the selected site into the patient's skin. The clinician continues to advance the device until venipuncture has been confirmed. This confirmation is usually done visually when the clinician sees blood entering a flashback chamber formed in the needle hub at the proximal end of the needle. After venipuncture has been confirmed, the clinician advances the sheath portion of the introducer distally into the patient's vein and the needle is withdrawn. With the catheter introducer properly placed, the clinician can then insert a catheter, guide wire or other relatively long, thin and flexible medical device, into the proximal opening of the sheath and continue to advance the catheter or other device through the catheter introducer until it is properly placed in the patient's vasculature. After placement of the catheter or other device, the clinician withdraws the introducer proximally out of the venipuncture site. The clinician then splits the introducer into its two separate halves by some combination of twisting and/or pulling apart of the wings along tear lines in the top and bottom walls of the introducer until the two halves of the introducer hub and sheath split apart.

When used on neonatal patients conventional over-the-needle peel-away catheter introducers are often awkward and difficult to employ. One critical concern is the insertion angle or angle of attack after initial piercing of the tissue, namely the angle of the introducer needle axis relative to the surface of the patient's skin during venipuncture. An infant's vein is so tiny and so close to the surface that with insertion proceeding at any typical insertion angle through the near wall of the vein, the needle could easily puncture the far wall of the vein.

Early prior art introducers were designed and developed for adults. Conventional introducers have structure which automatically forces venipuncture and advancement of the catheter to occur at a substantially higher angle than optimal for neonatal patients, because the construction of these introducers leads to a shape and thickness of the bottom of the introducer hub and of the bottom of the wings which causes the proximal part of the introducer to remain elevated and away from the surface of the skin. Such structure is not a problem with introducers for adults because the veins are far larger and more readily discernable and accessible. With advances in medical diagnosis and treatment, there is far greater application of venipuncture in neonatal patients, and thus there has developed a need for a catheter introducer that overcomes the above-described risks associated with venipuncture in neonatal patients or at least renders the venipuncture procedure safer, easier and quicker.

As limitations and/or problems became apparent when seeking to use these devices with infants, efforts at improvements began to appear. Disclosure in one prior art patent, U.S. Pat. No. 6,273,871, alleges to address and solve this problem, but ultimately does not. As seen in FIG. 2 and particularly in FIG. 3 of this patent, there is a substantial bulk of material in the hub 70 below the bottom of the sheath 60. The thickness of this material below the sheath means that the position of the introducer above the skin and the resultant insertion angle will always be substantial. This '871 patent alleges to reduce the insertion angle by altering the center of mass of the needle hub, whereas the present invention concerns the insertion angle primarily after the needle is removed, which is a different issue and requires a different approach.

U.S. Pat. No. 5,221,263 discloses another peel-away catheter where the hub 20 provides a full housing for the sheath's proximal end which is thus elevated and cannot lie flat on the patient's skin.

U.S. Pat. No. 5,334,157 discloses another catheter introducer which is a non-peel-away type, but which has a flat bottom. This device also has the sheath substantially elevated and within a separate housing.

The present invention employs a novel and substantially different construction which provides a new and improved catheter introducer along with a new method of making this catheter introducer and a new method of using it.

### SUMMARY OF THE INVENTION

It is an object of this invention to provide an over-the-needle peel-away sheath catheter introducer that allows a very low insertion angle during venipuncture. For convenience and simplification of terminology, references hereinafter to “catheter introducer” will mean “over-the-needle peel-away catheter introducer”.

It is another object of this invention to provide a catheter introducer that allows an insertion angle during advancement that is as low as one-two degrees for the introducer and needle combination and as low as zero degrees for the introducer without the needle.

It is a further object of this invention to provide a catheter introducer whose sheath is situated at the bottom of said introducer with the lumen of said sheath located above the bottom of the introducer by the wall thickness of said sheath.

In contrast to prior art catheter introducers formed of a hub housing about a sheath and its lumen, it is a further object of this invention to provide in the hub a lumen portion in substitution of the sheath and its lumen.

It is a still further object of this invention to provide a catheter introducer where the bottom of the sheath, the bottom of the wings and the bottom of the hub define a generally flat plane.

Another object of this invention is to provide a catheter introducer which includes a funnel element at the proximal end of the hub, where the bottom of the funnel is at substantially the same elevation as the bottom of the hub and the bottom of the wings.

An additional object of this invention is to provide a hub for a catheter introducer having a sheath therethrough, where the hub comprises left and right halves whose opposing side walls define sides of the lumen, and where the top and bottom walls of the sheath comprise material extending between said sides.

A further object of this invention is to form a catheter introducer hub, wings and funnel all as a contiguous molded body.

Another object of this invention is to provide non-peel-away catheter introducers with the features described above.

A still further object of this invention is to provide a method of injection molding the new catheter introducer employing a removable core pin that extends through the cavity forming the hub and through the lumen of a sheath whose proximal end is positioned axially within the mold cavity where the distal end of the hub will be formed, and where the hub halves and top and bottom walls are overmolded about the core pin which pin defines a lumen extending through the hub.

An additional object is to provide a method of introducing a catheter or guide wire into a vein of a patient by providing a catheter introducer whose thickness below the lumen is no more than the thickness of the lumen wall, so that the angle of insertion (or angle of attack) can be extremely low and essentially zero degrees after the introducer needle is removed and the introducer is being advanced into the vein. Here, the introducer is essentially parallel to or flat against the patient's skin, with the catheter spaced above the skin by only the wall thickness of the sheath. Furthermore, the preferred embodiment of the new introducer has an essentially flat bottom where the bottom surfaces of the sheath and of the hub and of the funnel define a plane which is at the lowest elevation of the introducer and which provides stability of the introducer from rocking from side to side or from rotation about a vertical axis.

The present invention thus comprises a new catheter introducer structure, a new method of manufacturing this introducer and a new method of introducing a catheter or guide wire using the new introducer. These three embodiments are exemplified by the descriptions presented below.

A first embodiment of the new catheter introducer comprises:
a. a hub having opposite proximal and distal ends, said hub comprising right and left halves extending axially between said proximal and distal ends, with a hub lumen extending axially between said halves,
b. a wing extending from each of said halves transversely of said hub lumen,
c. a funnel formed of converging cylindrical walls having a proximal end opening of first diameter and a distal end opening of diameter less than that of said first diameter, said distal end of said funnel joined to said proximal end of said hub, said funnel having a lumen communicating with said hub lumen, and
d. a sheath comprising a cylindrical wall formed as a tube having a proximal end joined to said distal end of said hub, a distal end and a sheath lumen which communicates with said hub lumen,
   said right and left halves of said hub having adjacent and spaced apart inner wall surfaces, said hub further comprising top and bottom walls extending between and joining said spaced apart inner wall surfaces, with said hub lumen being defined by said spaced apart inner wall surfaces and said top and bottom walls, said introducer including tear lines along the length of said funnel, hub and sheath for adapting said introducer to be split into two parts when said wings are pulled away from each other.

If desired, this structure may be employed with an introducer that does not include a funnel part or one where the funnel is incorporated into the hub instead of extending proximally from the hub.

The new invention further comprises a method of injection molding the new catheter introducer having a funnel entry part, a hub and a sheath and a continuous lumen therethrough, wherein said lumen in said hub is defined by left and right hub halves and top and bottom walls extending between and joining said halves, and wherein an axial extension of said sheath's proximal end is situated in the distal part of said hub and is coaxial and coextensive with said lumen in said hub, comprising the steps:
a. providing an injection mold with a cavity to define said funnel entry part and said hub,
b. positioning a sheath with its proximal end axially within the cavity where the distal end of the hub will be formed,
c. removably positioning a core pin axially through said cavity where the lumens of the hub and funnel entry parts will be situated and through said sheath lumen,
d. injecting plastic into said mold which plastic surrounds said core pin thus forming right and left halves of said hub, top and bottom walls joining said left and right halves, and a lumen in the space occupied by said core pin between said right and left halves and between said top and bottom walls, which lumen is coaxial and coextensive with said lumen in said proximal end of said sheath situated in said cavity, the plastic of said top and bottom walls and of said right and left halves bonding with the tubular end wall of said proximal end of said sheath, and
e. opening said mold and removing said core pin and removing said catheter introducer.

The new invention further includes a method of using the new introducer comprising the steps:
a. providing a catheter introducer comprising a funnel entry part, a central hub part and a distal sheath part, with a continuous lumen extending through said parts, where said introducer has a bottom surface which is the lowest part of said introducer, and where said continuous lumen is spaced from said bottom surface a distance no greater than the thickness of said lumen wall,
b. inserting an introducer needle through said continuous lumen until the distal end of said needle extends distally out of the distal end of said sheath,
c. positioning said catheter needle and catheter introducer for insertion,
d. piercing and dilating the skin and the near wall of the vein of the patient with the distal ends of said introducer needle and of said introducer sheath,
e. after initial piercing of the skin and of the near wall of the vein and flashback has been observed, reducing said insertion angle by lowering said introducer's proximal end until said bottom surface of said introducer lies generally parallel to the patient's skin, and advancing said needle and introducer sheath into said vein a predetermined distance, to ensure that the distal ends of both the introducer needle and catheter introducer sheath are in the patient's vein,
f. advancing said introducer sheath distally further into the vein and withdrawing said needle out of the introducer,
g. introducing a catheter or guide wire through said continuous lumen in said introducer and into the patient's vein,
h. withdrawing said introducer sheath proximally out of said vein and skin of the patient, and
i. splitting said introducer hub and funnel entry parts and peeling said introducer hub part and sheath into two parts and removing them from said catheter.

Further features and advantages of the invention as well as the structure and operation of various embodiments of the present invention are described in detail below with reference to the accompanying drawings.

### BRIEF DESCRIPTION OF THE DRAWINGS

FIG. 1 is a top perspective view of the new catheter introducer.

FIG. 2 is a top plan view thereof.

FIG. 3 is a left side elevational view thereof.

FIG. 4 is a top sectional view taken along line 4-4 in FIG. 3.

FIG. 5 is a sectional view taken along line 5-5 in FIG. 2.

FIG. 6 is a sectional view taken along line 6-6 in FIG. 2.

FIG. 7 is a sectional view taken along line 7-7 in FIG. 2.

FIG. 8 is a sectional view taken along line 8-8 in FIG. 2.

FIG. 9 is a front end elevational view of the introducer in FIG. 1.

FIG. 10 is a top perspective view of the new introducer with an introducer needle during initial venipuncture.

FIG. 11 is a view similar to FIG. 10, but at a further stage of venipuncture.

FIG. 12 is an elevational view of the new introducer during venipuncture.

FIG. 13 is a top perspective view of an inserted catheter after withdrawal and splitting of the introducer.

FIG. 14 is a schematic top plan view representing injection molding of the new introducer.

FIG. 15 is a sectional view taken along line 15-15 in FIG. 14.

### DETAILED DESCRIPTION OF THE PREFERRED EMBODIMENTS

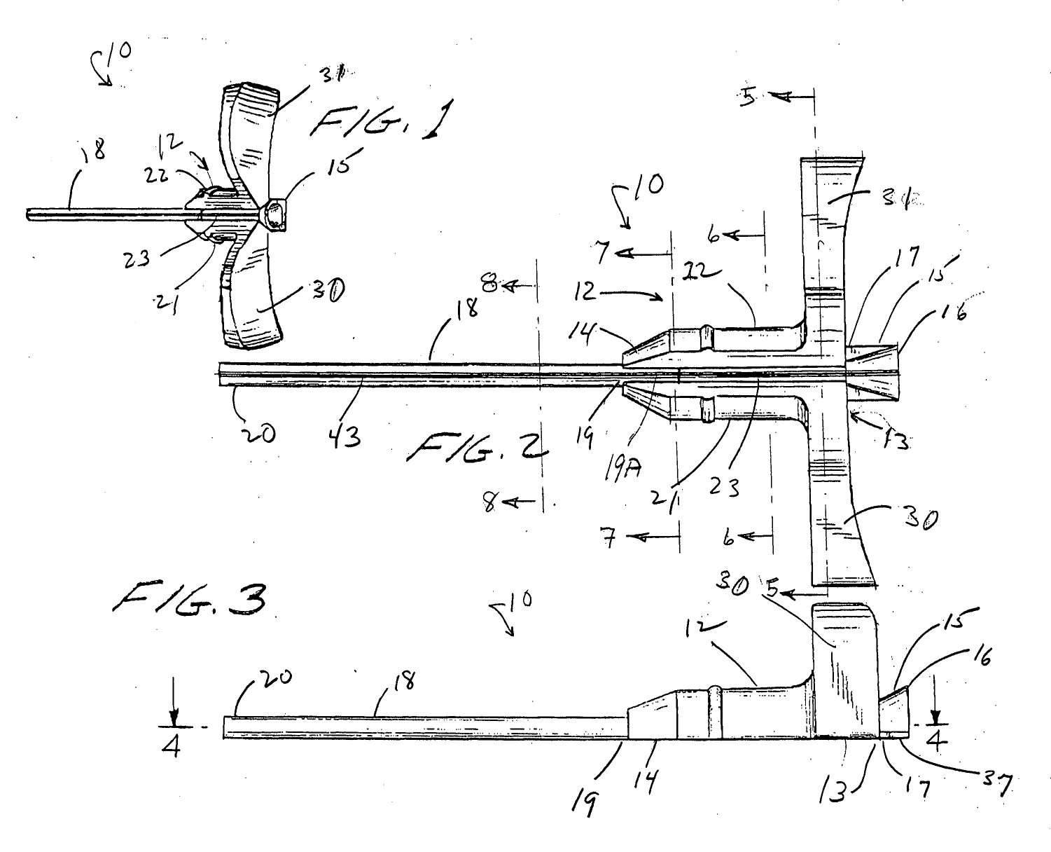

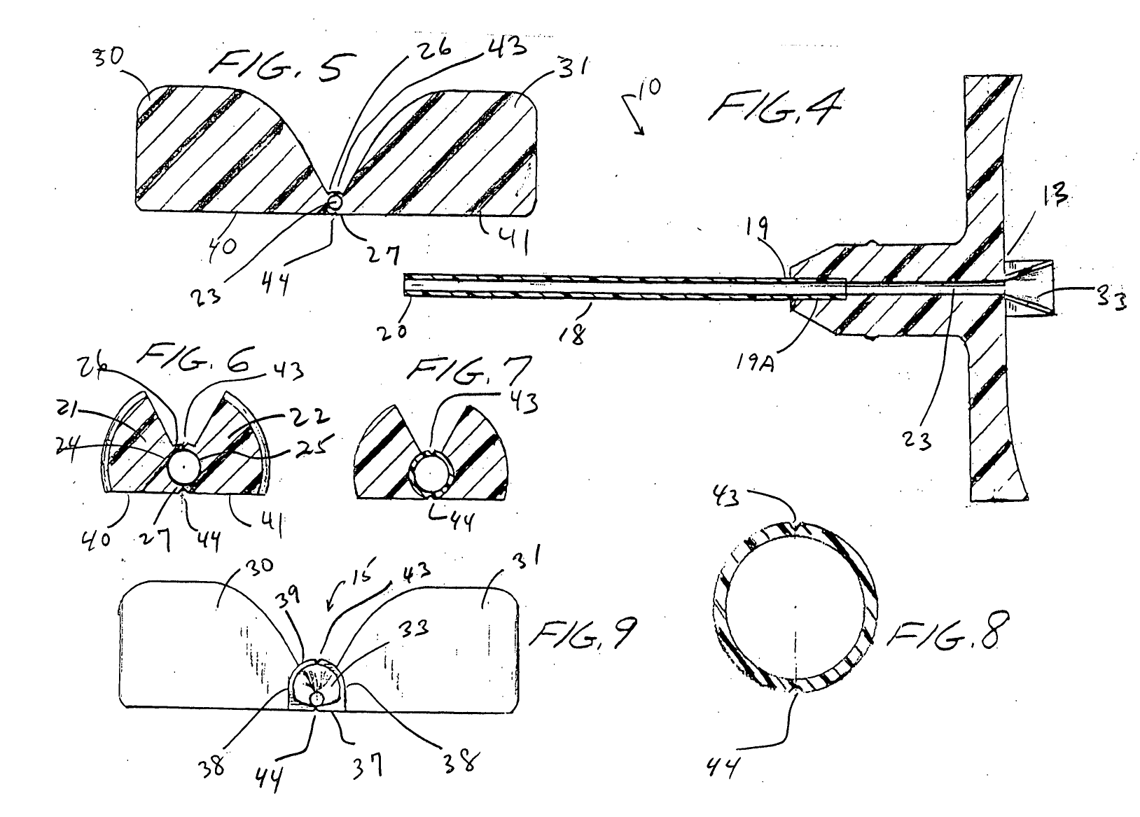

As seen in FIGS. 1-4 the new catheter introducer 10 comprises a hub 12 having proximal and distal ends 13, 14, a funnel 15 having proximal and distal ends 16, 17, and a sheath 18 having proximal and distal ends 19, 20.

The hub 12 is formed of opposite left and right halves 21, 22 with a lumen 23 between them. As seen in FIG. 1-6 the lumen 23 is defined by its side walls 24, 25 which are opposing surfaces of said left and right halves of the hub, and by top and bottom thin walls 26, 27 which extend between said halves.

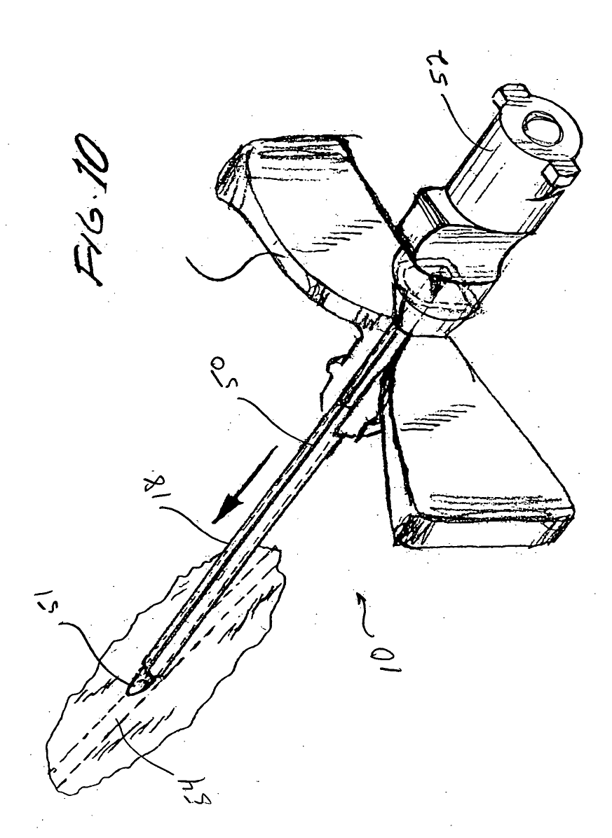

As seen in FIGS. 1-5, wings 30, 31 extend transversely from said hub halves. These wings are situated at the proximal end 13 of the hub. The wings 30, 31 comprise generally flat blades which curve downward as they approach hub halves 21, 22. The wings have substantially flat bottom surfaces 40, 41 as seen in FIGS. 5 and 6. The bottom surfaces of these wings and the bottom surface of hub 12 and of funnel 15 are all in substantially the same plane, representing the lowest part of the entire introducer.

Funnel 15 as seen in FIGS. 1-5 and 9 is a converging cylindrical body whose lumen 33 has a larger proximal end opening and a smaller distal end opening. The distal end 17 of funnel 15 is joined to the proximal end 13 of hub 12, with their respective lumens aligned and of the same diameter. Funnel 15 at its proximal end 16 defines in end view a generally flat bottom 37, generally parallel side walls 38 extending upward from said bottom, and a generally round top 39.

The sheath 18 is a hollow tube whose proximal end extension 19A, as seen in FIGS. 1, 2 and 4, is situated proximally inward of the distal end 14 of hub 12. The hub is molded onto the proximal end extension 19A of sheath 18, as described in greater detail in later sections herein.

As seen in FIGS. 4 and 6, sheath 18 is a tube formed of a cylindrical wall of generally uniform thickness. The top and bottom walls 26, 27 of the hub lumen 23 and said side walls 24, 25 thereof form a tube that is essentially an extension of the sheath, extending from proximal end extension 19A of the sheath to proximal end 13 of the hub. Said top and bottom walls 26, 27 have thickness essentially the same as said sheath wall thickness, which is conventionally about 0.006 inches.

The sheath is formed of suitable polymeric material, for example, high density polyethylene plastic, and is made by extrusion or other means prior to and independently of the molding of said hub. The hub halves 21, 22, wings 30, 31, top and bottom walls 26, 27 and funnel 15 are molded, also of suitable polymeric material, for example, high density polyethylene, in a single stage, where the hub halves 21, 22 are molded onto and bonded with the proximal end 19 of sheath 18. Typically, a radiopaque stripe is co-extruded along the length of the sheath.

In summary, the new introducer is injection molded as represented schematically in FIGS. 14 and 15 which show how the hub 12 is overmolded onto the sheath's proximal extension end 19A. Using conventional injection molding apparatus, the mold as represented schematically by the bottom and top halves 46, 46A of the mold, defines a cavity 47 for forming the hub, wings, and funnel shown by dashed reference lines to reference the numbers 12, 30, 31 and 15 respectively. Alternative side-by-side mold halves could be used. In the actual molding process the distal portion of core pin 48 is positioned in the lumen of the preformed sheath 18 to maintain its patency. The sheath then has its proximal end extension 19A positioned in the portion of the mold that forms the distal end of the hub, while the proximal portion of the core pin extends out of the sheath and through the hub area 12. Thereafter, molding the hub circumferentially about core pin 48 defines, upon removal of the pin, the hub lumen.

The core pin 48 is enlarged at its proximal end to define the diverging lumen of the funnel 15. In this manner the hub lumen 23 has internal diameter the same as that of the sheath 18 and of the distal end of the funnel 15.

As seen in FIG. 6, the hub lumen 23 is defined more particularly by opposing side walls 24, 25 of the hub halves 21, 22 and by top and bottom walls 26, 27 extending between and joining the hub halves of these walls being molded over the core pin 48 and over the proximal end extension 19A of sheath 18.

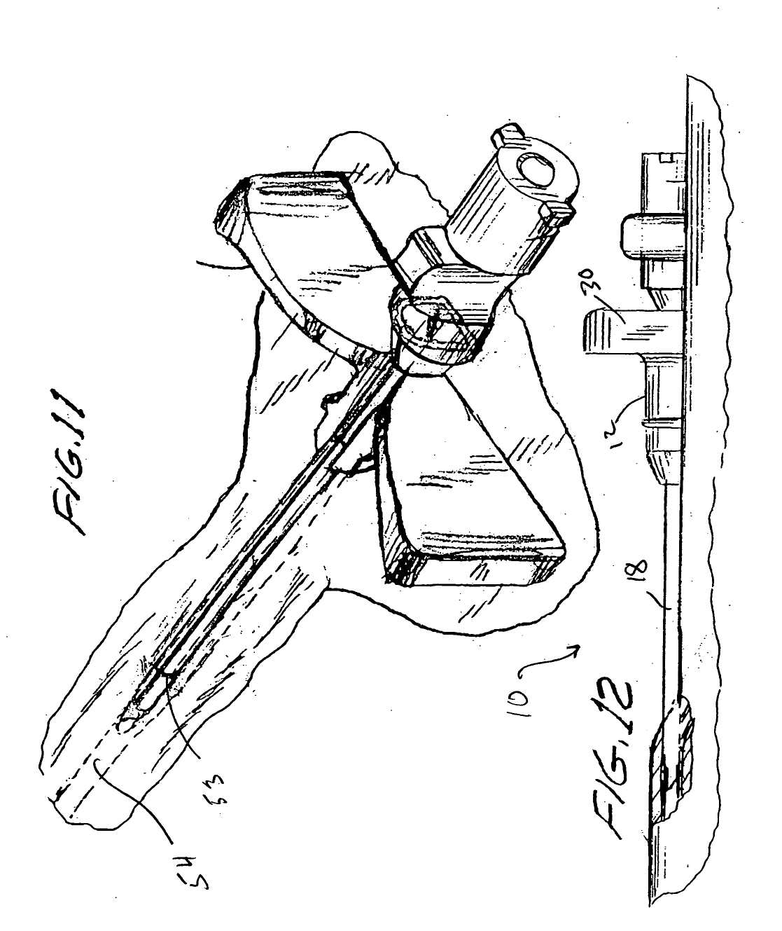

The final phase of manufacture of this peel-away catheter introducer is to form tear lines, 43, 44 in the center of the top and bottom surfaces, along the entire length of the sheath, hub and funnel, as seen in FIGS. 2 and 5-9. The tear lines can be made in a number of ways, including but not limited to, razor cuts and laser cuts. The depth of the cuts may be about 0.003 inches for certain neonatal introducers or commonly about fifty percent of the wall thickness, the guideline being that the material remaining after the cut should be strong enough to maintain the product's structure and function and weak enough to be readily tearable by clinicians using these products. The wall thicknesses of the sheath, of the hub and of the funnel are substantially the same, such that laser cutting of the tear lines can be uniform and swift, so that the resultant skived or scored walls will tear apart easily and uniformly along their length.

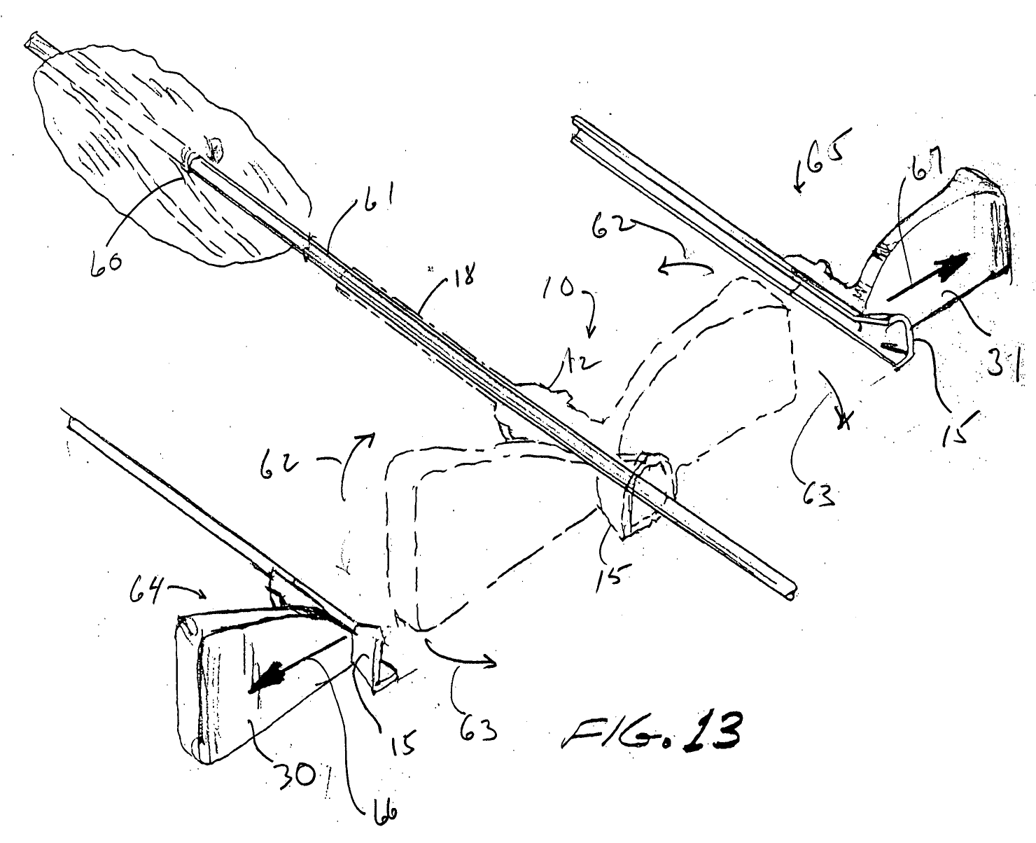

As seen in FIGS. 10 and 11, when venipuncture is initiated as introducer sheath 18 with needle 50 is directed toward the incision site, the skin is pierced and entry into a vein 54 begins. In accordance with the present invention, very promptly after entry of the distal ends of the needle and introducer sheath 50, 18, as seen in FIGS. 10 and 11, the introducer and needle combination is lowered from the initial angle of insertion to a very low angle or substantially parallel to or flat against the skin, as seen in FIG. 12, to allow advancement of the needle and sheath combination 50, 18 into the vein 54 followed by withdrawal of the needle. By this method and with this device, the risk of piercing the far wall of the vein is greatly reduced. The low angle is possible because the new structure has eliminated all material below the sheath, while still providing a hub and wings that are strong, stable and readily peelable at the appropriate time. More specifically, the overmolded hub defines bottom lumen walls no lower than the sheath's bottom walls and of substantially the same thickness as the sheath's bottom walls. The walls of the hub and sheath lumen, when similarly skived or scored, present the same tear strength characteristics. The needle or stylet 50 is well known in the prior art and is typically made of stainless steel with the needle's hub part 52 made of any suitable polymeric material, for example acrylic or K-resin.

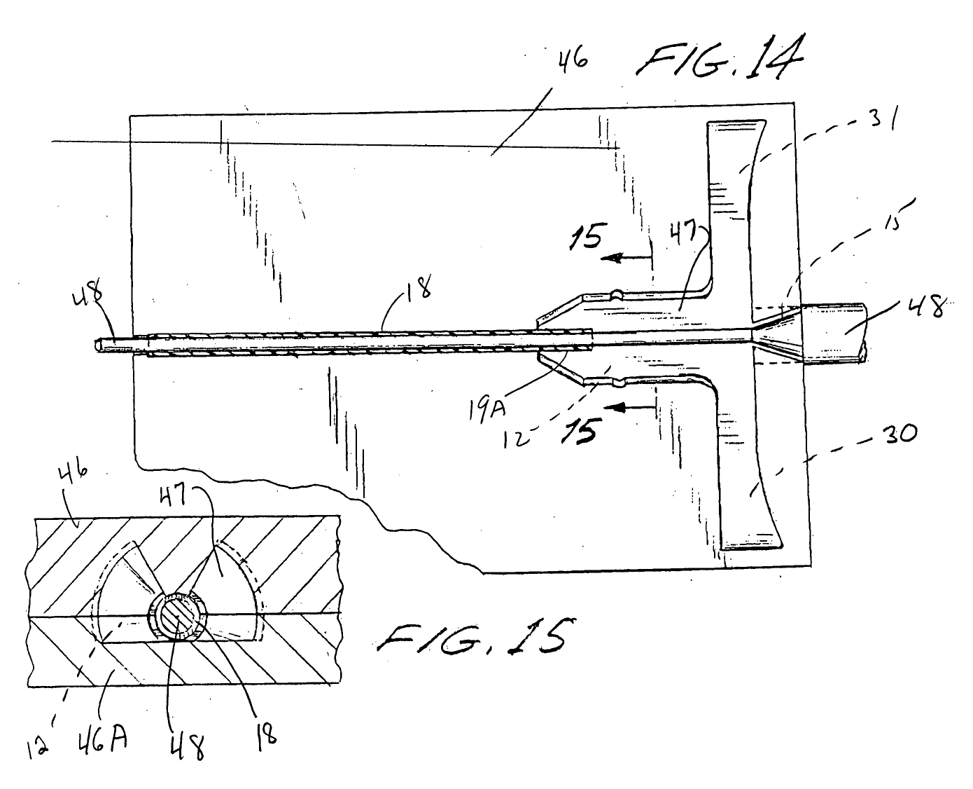

Following insertion of catheter 61 through the introducer into the vein, FIG. 13 illustrates withdrawal of the introducer sheath 18, shown in dashed lines, from the incision site 60 while the catheter 61 remains. In a conventional manner the wings 30, 31 are twisted upward and/or downward as indicated by arrows 62, 63 respectively until the sheath, hub and funnel 18, 12, 15 break apart along the top and bottom tear lines. The two halves of the introducer 64, 65 are shown separated and peeled apart when pulled in the directions of arrows 66 and 67.

While various embodiments of the present invention have been described above, it should be understood that they have been presented by way of example and not limitation. It will be apparent to persons skilled in the relevant art that various changes in form and detail can be made therein without departing from the spirit and scope of the invention. Thus, the present invention should not be limited by any of the above described exemplary embodiments, but should be defined only in accordance with the following claims and their equivalents.

1. A peel-away catheter introducer comprising:
a. a hub having opposite proximal and distal ends, said hub comprising right and left halves extending axially between said proximal and distal ends, with a hub lumen extending axially between said halves,
b. a wing extending from each of said halves transversely of said hub lumen,
c. a funnel formed of converging cylindrical walls having a proximal end opening of first diameter and a distal end opening of diameter less than that of said first diameter, said distal end of said funnel joined to said proximal end of said hub, said funnel having a lumen communicating with said hub lumen, and
d. a sheath comprising a cylindrical wall formed as a tube having a proximal end joined to said distal end of said hub, a distal end and a sheath lumen communicating with said hub lumen,
said right and left halves of said hub having adjacent and spaced apart inner wall surfaces, said hub further comprising top and bottom walls extending between and joining said spaced apart inner wall surfaces, with said hub lumen being defined by said spaced apart inner wall surfaces and said top and bottom walls, said introducer including tear lines formed along the length of said funnel, hub and sheath for adapting said introducer to be split into two parts when said wings are pulled away from each other.

2. A catheter introducer according to claim 1 wherein said hub, wings and funnel comprise contiguous molded plastic, said hub lumen being generally coaxial with and having substantially the same internal diameter as said sheath lumen.

3. A catheter introducer according to claim 1 wherein said funnel, hub and sheath lumens are substantially coaxial, and wherein said funnel, hub and sheath comprise contiguous central top wall parts and contiguous bottom wall parts respectively overlying and underlying said funnel, hub and sheath lumens.

4. A catheter introducer according to claim 3 where each of said contiguous top and bottom wall parts has an outer surface which includes therein said tear line cut in said outer surface and extending the length of said surface.

5. A catheter introducer according to claim 1 wherein said top and bottom walls of said hub each have generally uniform thickness.

6. A catheter introducer according to claim 3 wherein said hub has axial length between its proximal and distal ends, and wherein said top and bottom walls of said hub joining said hub halves have axial length greater than 50% of said axial length of said hub.

7. A catheter introducer according to claim 6 wherein said top and bottom walls of said hub and joining said hub halves have axial length greater than 70% of said axial length of said hub.

8. A catheter introducer according to claim 1 wherein said sheath's proximal end has an extension situated axially and partially within said hub's distal end.

9. A catheter introducer according to claim 8 wherein said hub is molded onto and bonded to said sheath's proximal end extension.

10. A catheter introducer according to claim 1 wherein said funnel's proximal end opening in cross-section defines a generally flat bottom wall, generally parallel side walls extending upward from said bottom wall and a generally round top wall.

11. A catheter introducer according to claim 1 wherein said funnel has a frustoconical shape.

12. A catheter introducer according to claim 1 wherein said sheath's cylindrical wall has a predetermined generally uniform thickness, and said top and bottom walls of said hub have thickness generally the same as the thickness of said sheath wall.

13. A catheter introducer according to claim I wherein each of said wings comprises an upstanding wall having a bottom surface no lower than the bottom wall of said hub.

14. A catheter introducer according to claim 13 wherein said bottom surfaces of said wings and said bottom walls of said hub, funnel and sheath respectively are generally coplanar.

15. A catheter introducer according to claim 1 wherein each of said wings is situated at said proximal end of said hub.

16. A catheter introducer according to claim 4 wherein said tear lines comprise cuts made in said outer surfaces of said top and bottom wall parts respectively.

17. A catheter introducer according to claim 4 wherein said contiguous central bottom wall parts of said funnel, hub and sheath are generally coplanar and are situated at the lowest elevation of the introducer.

18. A catheter introducer according to claim 17 wherein said contiguous bottom wall parts have generally uniform thickness.

19. A catheter introducer according to claim 1 wherein said sheath, hub, wings and funnel comprise a polymeric material.

20. A peel-away catheter introducer comprising:
a. a hub having opposite proximal and distal ends, said hub comprising right and left halves extending axially between said proximal and distal ends, with a hub lumen extending axially between said halves,
b. a wing extending from each of said halves transversely of said hub lumen, and
c. a sheath comprising a cylindrical wall formed as a tube having a proximal end joined to said distal end of said hub, a distal end and a sheath lumen communicating with said hub lumen,
said right and left halves of said hub having adjacent and spaced apart inner wall surfaces, said hub further comprising top and bottom walls extending between and joining said spaced apart inner wall surfaces, with said hub lumen being defined by said spaced apart inner wall surfaces and said top and bottom walls, said introducer including tear lines along the length of said funnel, hub and sheath for adapting said introducer to be split into two parts when said wings are pulled away from each other.

21. A kit comprising a catheter introducer according to claim 1 and an introducer needle with a sharp pointed distal end and an opposite proximal end, said needle adapted to be inserted into and through said funnel, through said hub and sheath, and to extend distally out of the distal end of said sheath for piercing the tissue and vein of a person for introduction of said introducer.

22. A peel-away catheter introducer comprising:
a. a hub having opposite proximal and distal ends, said hub comprising right and left halves extending axially between said proximal and distal ends, with a hub lumen extending axially between said halves,
b. a wing extending from each of said halves transversely of said hub lumen,
c. a funnel formed of converging cylindrical walls having a proximal end opening of first diameter and a distal end opening of diameter less than that of said first diameter, said distal end of said funnel joined to said proximal end of said hub, said funnel having a lumen communicating with said hub lumen, and
d. a sheath comprising a cylindrical wall formed as a tube having a proximal end joined to said distal end of said hub, a distal end and a sheath lumen communicating with said hub lumen,
said funnel, hub and sheath having contiguous top walls of generally uniform thickness and contiguous bottom walls of generally uniform thickness, with the outer surfaces of said bottom walls defining the bottommost part of said introducer,
said top and bottom walls defining between them a continuous lumen extending through said funnel, hub and sheath,
said continuous lumen being spaced from said bottom surface a distance no greater than said thickness of said bottom walls, and said top and bottom walls further including tear lines along their lengths for adapting said introducer to be split into two parts when said wings are pulled away from each other.

23. A peel-away catheter introducer comprising:
a. a hub having opposite proximal and distal ends, said hub comprising right and left halves extending axially between said proximal and distal ends, with a hub lumen extending axially between said halves,
b. a wing extending from each of said halves transversely of said hub lumen,
c. a funnel formed of converging cylindrical walls having a proximal end opening of first diameter and a distal end opening of diameter less than that of said first diameter, said distal end of said funnel situated at said proximal end of said hub, said funnel having a lumen communicating with said hub lumen, and
d. a sheath comprising a cylindrical wall formed as a tube having a proximal end joined to said distal end of said hub, a distal end and a sheath lumen communicating with said hub lumen,
said funnel, hub and sheath having contiguous top walls of generally uniform thickness and contiguous bottom walls of generally uniform thickness, with the outer surfaces of said bottom walls defining the bottommost part of said introducer,
said top and bottom walls defining between them a continuous lumen extending through said funnel, hub and sheath,
said continuous lumen being spaced from said bottom surface a distance no greater than said thickness of said bottom walls, and said top and bottom walls further including tear lines along their lengths for adapting said introducer to be split into two parts when said wings are pulled away from each other.

24. A method of introducing a catheter or guide wire into a vein of a patient, comprising the steps:
a. providing a peel-away catheter introducer comprising a funnel entry part, an introducer hub part and a distal sheath part, with a continuous lumen extending through said parts, where said introducer has a bottom surface which is the lowest part of said introducer, and where said continuous lumen is spaced from said bottom surface a distance no greater than the thickness of said lumen wall,
b. inserting an introducer needle through said continuous lumen until the distal end of said needle extends distally out of the distal end of said sheath,
c. positioning said introducer for insertion into the skin surface of the patient,
d. piercing and dilating the skin and the near wall of the vein of the patient with the distal ends of said needle and of said sheath,
e. after initial piercing of the skin and of the near wall of the vein and flashback has been observed, reducing said insertion angle by lowering said introducer's proximal end until said bottom surface of said introducer lies generally parallel to the patient's skin, and advancing said introducer's distal sheath part and said introducer needle into said vein a predetermined distance to ensure that both the introducer needle and introducer sheath are in the patient's vein,
f. advancing said introducer sheath distally further into the vein and withdrawing said needle from said introducer,
g. introducing a catheter or guide wire through said continuous lumen in said introducer,
h. withdrawing said introducer proximally out of said vein of the patient, and
i. splitting and peeling said introducer hub and funnel entry parts and sheath into two parts and removing them from said catheter.

25. A method of introducing a catheter or guide wire into a vein of a patient, comprising the steps:
a. providing a peel-away catheter introducer comprising an introducer hub part and a distal sheath part, with a continuous lumen extending through said parts, where said introducer has a bottom surface which is the lowest part of said introducer, and where said continuous lumen is spaced from said bottom surface a distance no greater than the thickness of said lumen wall,
b. inserting an introducer needle through said continuous lumen until the distal end of said needle extends distally out of the distal end of said sheath,
c. positioning said introducer for insertion into the skin surface of the patient,
d. piercing and dilating the skin and the near wall of the vein of the patient with the distal ends of said needle and of said sheath,
e. after initial piercing of the skin and of the near wall of the vein and flashback has been observed, reducing said insertion angle by lowering said introducer's proximal end until said bottom surface of said introducer lies generally parallel to the patient's skin, and advancing said introducer's distal sheath part and said introducer needle into said vein a predetermined distance to ensure that both the introducer needle and introducer sheath are in the patient's vein,
f. advancing said introducer sheath distally further into the vein and withdrawing said needle from said introducer,
g. introducing a catheter or guide wire through said continuous lumen in said introducer,
h. withdrawing said introducer proximally out of said vein of the patient, and
i. splitting and peeling said introducer hub part and sheath into two parts and removing them from said catheter.

26. A method of injection molding a peel-away catheter introducer having a funnel entry part, a hub and a sheath and a continuous lumen therethrough, wherein said lumen in said hub is defined by left and right hub halves and top and bottom walls extending between and joining said halves, and wherein an axial extension of said sheath's proximal end is situated in the distal part of said hub and is coaxial and coextensive with said lumen in said hub, comprising the steps:
a. providing an injection mold with a cavity to define said funnel entry part and said hub,
b. positioning a distal portion of a core pin within said sheath's lumen, while a proximal portion of said core pin remains exposed,
c. positioning the proximal end of said sheath and included core pin within the cavity where the distal end of the hub will be formed, with said proximal portion of said core pin being positioned in said cavity where said hub lumen will be formed,
d. injecting plastic into said mold which plastic surrounds said proximal portion of said core pin thus forming right and left halves of said hub, top and bottom walls joining said left and right halves, and a lumen in the space occupied by said proximal end of said core pin between said right and left halves and between said top and bottom walls, which lumen is coaxial and coextensive with said lumen in said proximal end of said sheath situated in said cavity, plastic of said top and bottom walls and of said right and left halves bonding with the tubular end wall of said proximal end of said sheath, and
e. opening said mold and removing said core pin.

27. A peel-away catheter introducer comprising:
a. a hub having opposite proximal and distal ends, said hub comprising right and left halves extending axially between said proximal and distal ends, with a hub lumen extending axially between said halves,
b. a wing extending from each of said halves transversely of said hub lumen,
c. a funnel formed of converging cylindrical walls having a proximal end opening of first diameter and a distal end opening of diameter less than that of said first diameter, said funnel situated at said proximal end of said hub, said funnel having a lumen communicating with said hub lumen, and
d. a sheath comprising a cylindrical wall formed as a tube having a proximal end joined to said distal end of said hub, a distal end and a sheath lumen communicating with said hub lumen,
said right and left halves of said hub having adjacent and spaced apart inner wall surfaces, said hub further comprising top and bottom walls extending between and joining said spaced apart inner wall surfaces, with said hub lumen being defined by said spaced apart inner wall surfaces and said top and bottom walls, said introducer including tear lines formed along the length of said funnel, hub and sheath for adapting said introducer to be split into two parts when said wings are pulled away from each other.
